(19) United States
(12) Patent Application Publication
(10) Pub. No.: US 2022/0061906 A1
(43) Pub. Date: Mar. 3, 2022

(54) SURGICAL DEVICE CONNECTION HUB

(71) Applicant: GYRUS MEDICAL LIMITED, Cardiff (GB)

(72) Inventors: Wilhelmus GOMMEREN, Cardiff (GB); Nathan Philip BLAKE, Cardiff (GB); Rhydian HODDINOTT, Cardiff (GB)

(73) Assignee: GYRUS MEDICAL LIMITED, Cardiff (GB)

(21) Appl. No.: 17/391,147

(22) Filed: Aug. 2, 2021

(30) Foreign Application Priority Data
Aug. 26, 2020 (GB) 2013392.2

(51) Int. Cl. A61B 18/12

(52) U.S. Cl. CPC A61B 18/1206; A61B 2018/00178

(57) ABSTRACT

A surgical instrument which is configured to be releasably connected to a hand-piece is disclosed. The surgical instrument includes an elongate shaft, an end effector at a distal end of the elongate shaft, and a connection hub at a proximal end of the elongate shaft. The connection hub includes a moveable member moveable relative to the elongate shaft. The moveable member is configured to be switchable between an on position and an off position. During insertion of the surgical instrument into the hand-piece, an electrical connection therebetween is prevented by the connection hub when the moveable member is in the on position.

### TECHNICAL FIELD

Embodiments of the present invention described herein relate to a surgical device, and in particular to an electrosurgical device wherein a disposable instrument is connectable to a hand-piece via a connection hub, the connection hub facilitating an electrical and mechanical connection therebetween.

### BACKGROUND OF THE INVENTION

Electrosurgical instruments provide advantages over traditional surgical instruments in that they can be used for coagulation and tissue sealing purposes. One such prior art arrangement is known from U.S. Pat. No. 5,904,681 which describes a surgical instrument including a mechanical cutting portion, such as a rotary blade or burr, and a radio frequency (RF) cutting and/or cauterizing portion comprising an electronic surgical device which operates in bipolar mode.

Another prior art arrangement is known from U.S. Pat. No. 9,017,851 which describes an apparatus for powering a medical device, including a protective layer covering a battery pack and a connection feature. The connection feature provides a fluid tight seal, for example by having an electrode which may pierce the protective layer to establish electrical communication with the medical device from within the protective layer, thereby allowing a non-sterile battery pack to deliver power to a sterile medical device.

Another prior art arrangement is known from EP0746251B1 which describes an integrated catheter assembly including a catheter having an irrigation fluid lumen, a distal tip portion having electrodes, and a needle extending through the catheter lumen and a lumen in the distal tip portion. The catheter is carried by a needle hub structure which provides a sealing entrance for a needle that can be displaced between extended and retracted positions while being electrically isolated.

Another prior art arrangement is known from US2009/221955A1 which describes an ablative apparatus having a distal end at which an ablation probe driven by a transducer may be vibrated to ablate tissues.

### SUMMARY OF THE INVENTION

Embodiments of the present invention provide an improved surgical instrument having an elongate shaft comprising a connection hub and an end effector. The end effector may comprise a known end effector capable of different operations, including mechanical cutting or shaving of tissue ,and/or electrosurgical ablation, sealing and/or coagulation of tissue. The surgical instrument can be connected to a hand-piece for provision of fluid, mechanical power and/or radiofrequency (RF) electrical signals. A single hand-piece can be re-used for multiple surgeries while the surgical instrument may be disposable, meaning that it might only be used for a single surgical operation performed on a single patient, before being disposed. The focus of the disclosure is on the connection between the hand-piece and the surgical instrument, which must be capable of transferring mechanical as well as electrical power. The instrument disclosed herein facilitates the inclusion of electrical connections in the connection hub so that it may receive electrical signals and power from the hand-piece, while ensuring safe operation of the device. In particular, the connection hub has a static part that is fixed to the elongate shaft and a moveable member which can move, such as by rotation, relative to the elongate shaft. The moveable member may be a switch which can be moved between an on position and an off position in order to control the position of electrical contacts on the connection hub. During insertion of the surgical instrument into the hand-piece, the hand-piece has a safety feature which can block the moveable member when it is in the on position. Therefore, should a user attempt to slide or otherwise insert the instrument into the hand-piece while the switch is in the on position, the safety feature prevents the user from doing so. This prevents damage to the electrical connections and helps to avoid accidental activation of the surgical features. This safety feature can include a mutually cooperating interface between the hand-piece and the surgical instrument. The mutually cooperating interface can prevent the surgical instrument from being inserted into the hand-piece such that the surgical device would be in a live position. Therefore, should a user attempt to insert the instrument into the hand-piece in such a way that would result in the surgical device being activated, they will be prevented from doing so by virtue of the mutually cooperating interface, in order to prevent a potentially hazardous or damaging situation from occurring.

In view of the above, from one aspect the present invention provides a surgical instrument, configured to be releasably connectable to a hand-piece, the surgical instrument comprising:
an elongate shaft,
an end effector at a distal end of the elongate shaft, and
a connection hub at a proximal end of the elongate shaft, the connection hub comprising a moveable member, moveable relative to the elongate shaft, and configured to be switchable between an on position and an off position,
configured such that during insertion of the surgical instrument into the hand-piece, an electrical connection therebetween is prevented by the connection hub when the moveable member is in the on position.

Such an arrangement improves on those known in the art by providing a surgical instrument whose electrical connections are included in a connection hub instead of, for example, requiring a separate external power cable. This provides a more compact device that can be operated more easily by a user. This advantage is realised without compromising safety. In particular, the device provides a mutually cooperating interface which prevents insertion of the instrument into the hand-piece in a live state. Such prevention can be achieved by the moveable member on the connection hub which prevents it from being connected to the hand-piece when in its on position.

The moveable member may comprise an arm. The arm, in combination with a guard tab of the hand-piece, may be configured to prevent insertion of the surgical instrument into the hand-piece when the moveable member is in the on position. This can prevent an electrical connection between the surgical instrument and the hand-piece from being established, in order to prevent a live state of the device being activated immediately upon full insertion.

The connection hub may comprise at least one electrical contact. The at least one electrical contact may be configured to be aligned with at least one corresponding electrical contact on the hand-piece when the moveable member is in the on position. The surgical instrument may be configured such that after insertion of the surgical instrument into the hand-piece, switching the moveable member from the off position to the on position brings at least one electrical contact of the connection hub into alignment with at least one corresponding electrical contact of the hand-piece, to thereby permit electrical connection therebetween. This provides one possible way of the on and off positions of the moveable member being capable of respectively providing or preventing an electrical connection between the hand-piece and the instrument.

The connection hub may comprise a static member in fixed relation to the elongate shaft. The static member may comprise at least one locking tab. The at least one locking tab may be configured to be received by at least one locking slot of the hand-piece. This has the advantage of providing an additional stage of connection that may require the surgical instrument to be in a particular orientation before beginning to be inserted into the hand-piece.

The moveable member may comprise a cantilever. The cantilever may comprise a cantilever head. The static member may comprise a first groove which may be configured to receive the cantilever head. The static member may comprise a second groove which may be configured to receive the cantilever head. The moveable member may be configured such that switching the moveable member between the on position and the off position moves the cantilever head from the first groove to the second groove in order to provide two distinct switching positions of the moveable member. This has the advantage of limiting the movement of the moveable member and providing two, for example, distinct switching positions wherein feedback is provided to the user upon switching.

An aspect of the present invention provides a hand-piece, configured to power a surgical instrument, the hand-piece comprising:
a main body, and
a guard member in fixed relation to the main body, the guard member comprising a guard tab configured to prevent insertion of a moveable member of the surgical instrument into the hand-piece when the moveable member is in an on position,
wherein the moveable member is switchable between the on position and an off position.

The guard tab may comprise a cam surface. The cam surface may be configured to direct the moveable member of the surgical instrument towards the off position.

The hand-piece may comprise a collet. The collet may be rotatable with respect to the guard member and the main body between a locked and an unlocked position. The collet may be configured to prevent insertion of the surgical instrument into the hand-piece when in the locked position. The collet may be configured to switch the moveable member to its off position when the collet is rotated in a first direction. The collet may be configured to switch the moveable member to its on position when the collet is rotated in a second direction, opposite the first direction.

An aspect of the present invention provides a surgical device comprising a surgical instrument and a hand-piece as described hereinabove.

An aspect of the present invention provides a surgical system comprising an electrosurgical generator and the surgical device as described hereinabove. The electrosurgical generator may be comprised within the hand-piece as an integral part thereof.

An aspect of the present invention provides a surgical device comprising:
a hand-piece;
a surgical instrument comprising an elongate shaft and an end effector, the surgical instrument configured to be releasably connectable to the hand-piece; and
a mutually cooperating interface between the hand-piece and the surgical instrument configured to prevent insertion of the surgical instrument into the hand-piece in a live state.

### BRIEF DESCRIPTION OF THE DRAWINGS

Further features and advantages of the present invention will become apparent from the following description of embodiments thereof, presented by way of example only, and by reference to the drawings, wherein:

FIG. 1 is a schematic diagram of a surgical system including a surgical instrument according to an embodiment of the present invention.

FIG. 2 is a perspective view of a surgical system according to an embodiment of the present invention.

FIG. 3 is a further perspective view of a surgical system according to an embodiment of the present invention.

FIG. 4 is a perspective view of a hand-piece of a surgical system according to an embodiment of the present invention.

FIG. 5 is a perspective view of a surgical instrument according to an embodiment of the present invention.

FIG. 6A is a cross sectional view of a surgical instrument according to an embodiment of the present invention.

FIG. 6B is a further cross sectional view of a surgical instrument according to an embodiment of the present invention.

FIG. 7 is a perspective view of a hand-piece of a surgical system according to an embodiment of the present invention.

FIG. 8 is a perspective view of a surgical instrument according to an embodiment of the present invention.

FIG. 9 is a perspective view of a surgical system according to an embodiment of the present invention.

FIG. 10 is a top view of a surgical system according to an embodiment of the present invention.

FIG. 11 is a perspective view of a surgical system according to an embodiment of the present invention.

### DETAILED DESCRIPTION

A surgical instrument is described herein in the context of a surgical system for performing procedures such as ablation, sealing, resection and coagulation of tissues. The apparatus includes an electrosurgical generator capable of providing a mechanical and/or RF output. Such outputs may be controlled by various user inputs such as push buttons and switches, with the levels of various settings and other information being indicated on a display. The generator is connected to an electrosurgical device via a connection cord capable of transmitting power and RF signals thereto. Additionally, an irrigation and suction source is provided, capable of drawing fluid from, or providing fluid to, the electrosurgical device through tubes. The connection cord and tubes are connected to a hand-piece of the electrosurgical device. The hand-piece provides a means with which a practitioner may manipulate the electrosurgical device, and it may have further user input devices thereon for controlling the mechanical and/or RF output of the generator.

The surgical instrument, which is described herein by way of example as an electrosurgical instrument, can be releasably connected to the hand-piece. At a proximal end of the electrosurgical instrument there is a connection hub. The connection hub can include a switch mechanism that may be switchable between an on position and an off position. The switching mechanism of the connection hub may include a moveable member that is moveable with respect to the elongate shaft of the electrosurgical instrument. For example, the moveable member may be configured to be rotatable about an axis of the elongate shaft between the on position and the off position. The connection hub may comprise at least one electrical contact in fixed relation to the moveable member, such that switching the moveable member between its on and off positions changes the positions of the at least one electrical contact relative to the elongate shaft of the electrosurgical instrument.

The electrosurgical instrument may be slideably receivable by the hand-piece such that it can be inserted therein. The insertion can be separated into two stages of connection. The first stage can ensure that the instrument is inserted into the hand-piece in an appropriate position, such as at a particular angle relative to the hand-piece. In this respect, there may be provided a ‘poka-yoke’ feature, such as a lock-and-key arrangement, between the hand-piece and the instrument. This can ensure that the instrument is inserted correctly into the hand-piece, and may include at least one tab on the connection hub which is receivable by at least one slot of the hand-piece. The system can be configured such that the tabs may only be inserted into the corresponding slots when they are aligned. In this way, the first stage of connection dictates the relative positions of the instrument and the hand-piece to allow connection therebetween. The hand-piece may also comprise a locking mechanism which requires a user to move, such as by rotation, a portion of the hand-piece before the tabs can be fully inserted into the slots. The locking mechanism can have a locked position, in which the tabs may not enter or exit the slots, and an unlocked position, in which the tabs may enter or exit the slots. The locking mechanism may be actuated for example by rotation of a collet on the hand-piece. The locking mechanism may be biased towards a locked position such that after insertion of the tabs into the slots, releasing the collet allows the locking mechanism to be moved towards the locking mechanism to thereby lock the tabs in the slots and therefore lock the instrument to the hand-piece. In this arrangement, the instrument may be released from the hand-piece by unlocking the locking mechanism and removing the instrument from the hand-piece.

The first stage described above permits or prohibits insertion of the instrument into the hand-piece based upon a relative position, such as an angular alignment, of the instrument. Once the first stage of connection has been completed, i.e. the tabs have been aligned with and inserted into the slots, the elongate shaft of the instrument will be restricted to movement only in an axial direction. The second stage of connection requires that the switch of the instrument is in the correct position, such as the off position, before it can be fully inserted into the hand-piece. This can dictate that electrical contacts on the instrument be misaligned with electrical contacts on the hand-piece upon insertion. This prevents a potentially hazardous situation in which the instrument is inserted into the hand-piece while in the on position. The second stage of connection can be facilitated by an arm on the moveable member of the connection hub which can interact with a guard tab of the hand-piece. Specifically, when the moveable member is in the on position, it will be aligned with the guard tab of the hand-piece such that further insertion into the hand-piece is prevented. In contrast, when the moveable member is in the off position, the moveable member can be misaligned with the guard tab such that the instrument may be fully inserted into the hand-piece. Once fully inserted, the moveable member may be at an axial position beyond that of the guard tab, such that the switch may be moved to its on position to align the electrical contacts of the electrosurgical instrument with those of the hand-piece in order to permit operation of the electrosurgical system.

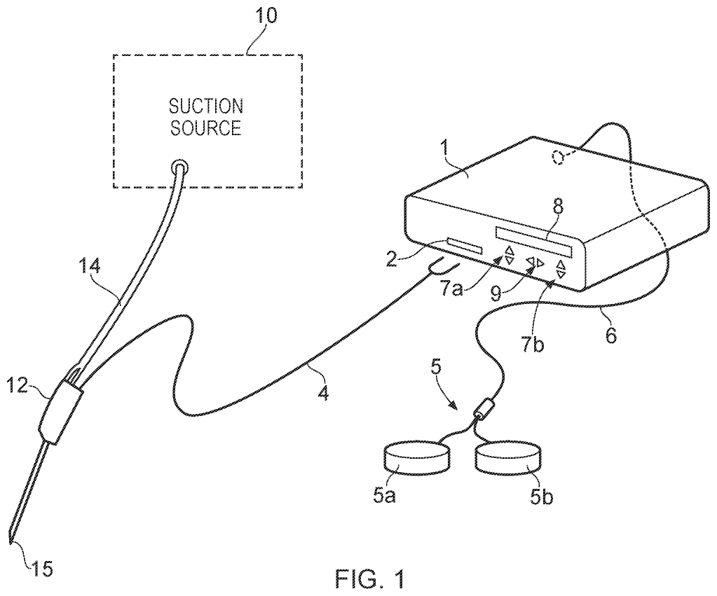

Referring to the drawings, FIG. 1 shows electrosurgical apparatus including an electrosurgical generator 1 having an output socket 2 providing a radio frequency (RF) output, via a connection cord 4, for an electrosurgical device 12. The device 12 has an end effector 15, and irrigation and suction tubes 14 which are connected to an irrigation fluid and suction source 10. Activation of the generator 1 may be performed from the device 12 via a hand-switch (not shown) on the device 12, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively, although in some embodiments of the electrosurgical device 12 described herein it is envisaged that only one or other of the coagulation or ablation modes would be used, with cutting being provided mechanically by way of a rotating tube having a sharpened cut-out portion. The generator front panel has push buttons 7a, 7b for respectively setting ablation (cutting) or coagulation power levels, which are indicated in a display 8. Push buttons 9 are provided as an alternative means for selection between the modes of operation. In an alternative arrangement, the device 12 may include means to generate electrosurgical signals, for example using an electrosurgical generator that can be an integral part of the device 12.

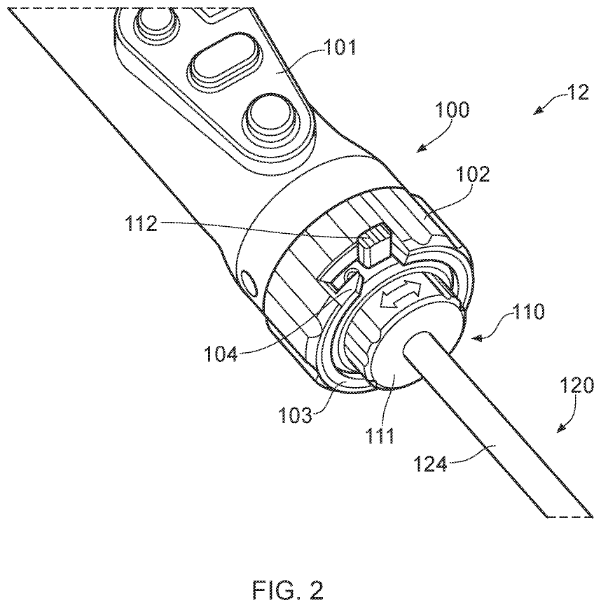

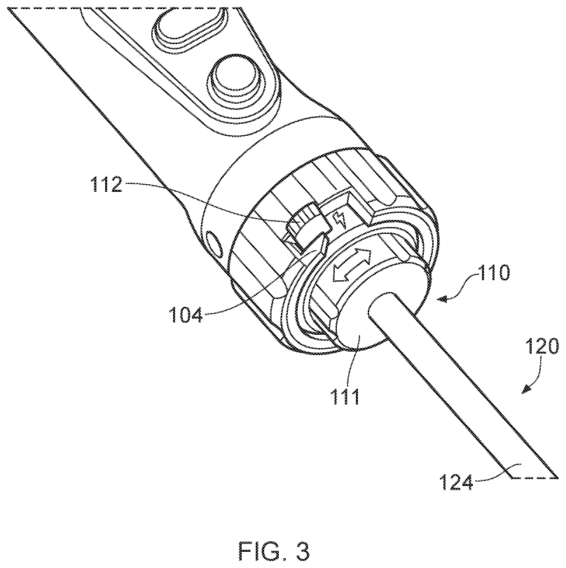

FIGS. 2 and 3 show part of the electrosurgical device 12 that forms the basis of an embodiment of the present disclosure. The electrosurgical device 12 comprises a hand-piece 100 and an electrosurgical instrument 120, shown to be fully inserted into the hand-piece 100. The hand-piece 100 comprises a main body 101 having activation buttons thereon to permit an operator to activate the mechanical cutting or electrosurgical operations of the device. The electrosurgical instrument 120 comprises an elongate shaft 124 having an end effector 15 at a distal end and a connection hub 110 at a proximal end. The connection hub 110 comprises a moveable member 111, which is moveable relative to the elongate shaft 124. In the example shown, the moveable member 111 is rotatable relative to the elongate shaft 124 about a longitudinal axis thereof.

The hand-piece 100 comprises a collet 102 and a guard member 103 located at a proximal end of the hand-piece 100. The collet 102 is substantially annular and extends from the hand-piece in a longitudinally axial direction therefrom. The guard member 103 is provided in fixed relation to the main body 101 of the hand-piece 100 and comprises a substantially annular portion received by the collet 102. The collet 102 is rotatable relative to the hand-piece 100 and the guard member 103. The collet 102 and the guard member 103 each define a window, being a gap in the circumferential wall of each component. The guard member 103 comprises a guard tab 104 which extends circumferentially into a portion of the window thereof of the guard member 103.

In FIGS. 2 and 3, the electrosurgical instrument 120 is shown to be inserted into the hand-piece 100 via its connection hub 110. A portion of the connection hub 110 is received by an axial chamber 107 defined within the main body 101, collet 102 and the guard member 103. The moveable member 111 of the connection hub 110 comprises an arm 112. The arm 112 is fixed to the moveable member 111 to control movement thereof. When the electrosurgical instrument 120 is fully inserted into the hand-piece as shown, the arm 112 of the moveable member 111 can be received by the windows comprised in the collet 102 and the guard member 103. The moveable member 111 can be switched between a plurality of positions by moving it relative to the elongate shaft 124. In the example shown, the moveable member 111 can be actuated by rotation between an on position and an off position. This may be achieved by applying a force to the arm 112. In FIG. 2, the moveable member is shown in its off position. In the off position, the arm 112 is misaligned with the guard tab 104 such that the electrosurgical instrument 120 can be removed by sliding it out of the channel 107 in the hand-piece 100 in an axial direction. In FIG. 3, the moveable member 111 is shown in its on position. In the on position, the arm 112 is aligned with the guard tab 104 such that the electrosurgical instrument 120 cannot be removed from the hand-piece, by virtue of the guard tab 104 restricting axial movement of the moveable member 111.

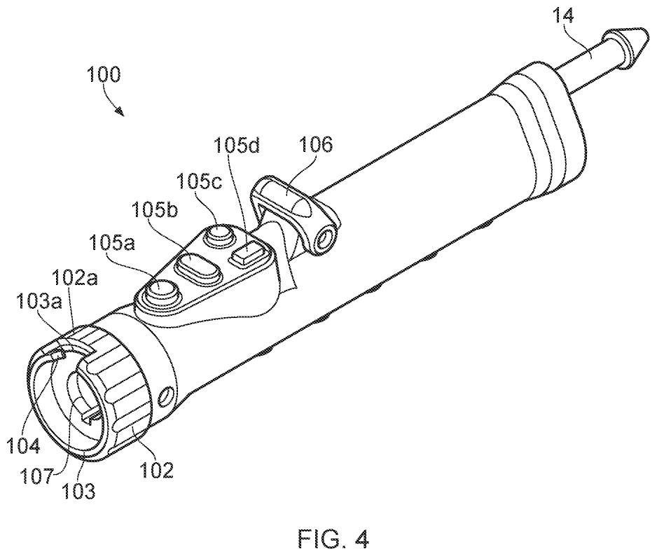

FIG. 4 shows further detail of the hand-piece 100 of the electrosurgical device 12, without the electrosurgical instrument 120 inserted therein. The hand-piece 100 may comprise a connector 14 at a distal end thereof for provision of power, electrosurgical signals, suction and/or irrigation. In addition to one or more buttons 105a, 105b, 105c, 105d, the main body 101 of the hand-piece 100 may comprise an input member 106, which may be a proportional switch or trigger. The collet 102 comprises a window 102a which may be provided as a recess in a portion of the circumferential wall of the collet 102. The guard member 103 comprises a window 103a which may be provided as a recess in the annular portion thereof. The guard tab 104 can be provided as a circumferential projection of the guard member 103.

As shown in FIGS. 2 to 4, the guard tab 104 projects from a circumferential edge of the window 103a into the gap defined by the window 103a. More specifically, at the proximal end of the hand-piece 100, the window 103a is comprised by the annular portion of the guard member 103 having a lower angular extent than that of the remainder of the guard member 103. This provides a cut-out portion in the guard member 103, wherein the window 103a is defined. At an axial face, the guard member comprises an annular portion having a higher angular extent than the portion that defines the window 103a, thereby providing the guard tab 104 which extends partially across the window 103a. For example, the guard member 103 may have an annular portion defined around the entire angular extent, i.e. 360°, of axis of the chamber 107. At an axial position closer towards the proximal end of the hand-piece 100, the guard tab 103 may have an annular portion defined around approximately 340° of the chamber 107, thereby defining the window 103a having an angular extent of approximately 20°. Then, at an axial position closer still towards the proximal end of the hand-piece 100, the guard tab 103 may have an annular portion defined around approximately 350° of the chamber 107, thereby defining the guard tab 104 having an angular extent of approximately 10°, leaving an angular gap of approximately 10° through which the arm 112 may pass.

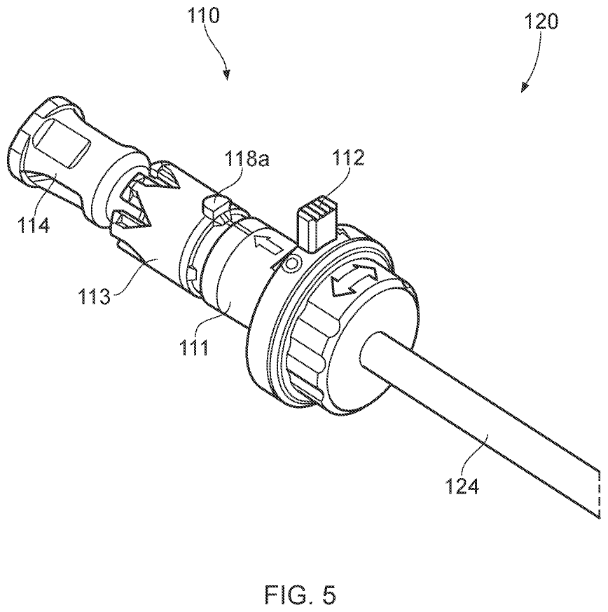

FIG. 5 shows part of the electrosurgical instrument 120. The connection hub 110 is comprised at a proximal end of the electrosurgical instrument 120. The connection hub 110 may be disposed circumferentially around a portion of the elongate shaft 124. The connection hub 110 comprises a static member 113 in fixed relation to the elongate shaft 124. In particular, the static member 113 is unable to rotate relative to the elongate shaft 124. The static member 113 comprises at least one locking tab 118a which may project from an outer circumferential surface of the static member 113. The electrosurgical instrument can also comprise an interface portion 114 which may form a mechanical and/or fluidic connection with corresponding portions (not shown) of the hand-piece 100, in order to provide motive power, suction and/or irrigation to the electrosurgical instrument 120.

As shown in FIG. 5, the connection hub 110 further comprises the moveable member 111 which is moveable relative to the elongate shaft 124. In particular, the moveable member 111 may rotate relative to the elongate shaft 124. The moveable member 111 is switchable between a plurality of positions and can be rotated relative to the elongate shaft 124 in order to switch it between the plurality of positions. The arm 112 is fixed to the moveable member 111 and may be an outward radial projection of the moveable member. The arm 112 provides a lever with which to switch the moveable member 111 between its plurality of positions. In this way, a force applied to the arm 112 in a suitable direction can rotate the moveable member 111 relative to the elongate shaft 124 and the static member 113 in order to switch between the switching positions.

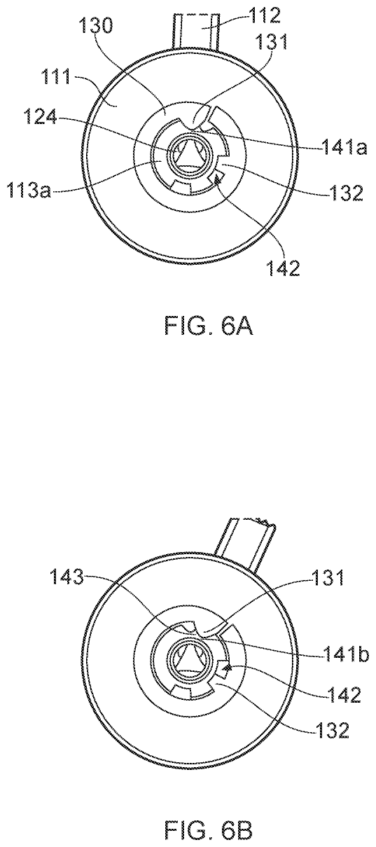

FIGS. 6A and 6B show a cross section of the electrosurgical instrument 120. The cross sections reveal the mechanism by which the moveable member 111 can be switched between its plurality of switching positions. An inner portion 113a of the static member 113 is disposed within the moveable member 111. A proximal portion of the elongate shaft 124 can also be seen to be disposed within the inner portion 113. The moveable member 111 comprises a cantilever 130 in fixed relation thereto. The cantilever is moveable with the moveable member 111 relative to the elongate shaft 124 and the static member 113. In the example shown, the cantilever 130 is rotatable with the moveable member 111 relative to the elongate shaft 124 and the inner portion 113a. The cantilever 130 may be positioned around the inner portion 113a such that it can exert a radially inward biasing force thereon. The cantilever comprises a head 131. The head 131 can be provided as a ridge or bump projecting radially inwards from the cantilever 130. The moveable member 111 further comprises a switching tab 132. The switching tab 132 can be provided as a ridge projecting radially inwards from the moveable member 111. In the arrangement shown, the switching tab 132 is comprised as an integral component of the cantilever 130.

The inner portion 113a of the static member 113 comprises a cylindrical body disposed around the elongate shaft 124 and received by the moveable member 111. The static member 113 comprises a slot 142 on an outer circumferential surface of the inner portion 113a. The slot 142 is configured to receive the switching tab 132. Specifically, the slot 142 is positioned so as to receive the switching tab 132 and has dimensions corresponding to those of the switching tab 132. The angular extent of the slot 142 around the elongate shaft 124 is greater than that of the switching tab 132. In other words, the slot 142 has a greater width than the switching tab 132 such that the switching tab 132 may be received in a plurality of positions in the slot 142. The slot 142 can restrict the degree of movement of the switching tab 132. For example, in FIG. 6A, the cantilever 130 is blocked from rotating in the anticlockwise direction by virtue of the tab 132 being restricted by a first wall of the slot 142. Similarly, when the moveable member 111 is in the position shown in FIG. 6B, the cantilever 130 is blocked from rotating in the clockwise direction by virtue of the switching tab 132 being restricted by a second wall of the slot 142. In this way, the movement of the moveable member 111 can be constrained.

The inner portion 113a further comprises a plurality of grooves. In the example shown, the inner portion 113a comprises a first groove 141a and a second groove 141b adjacent to the first groove 141a. The grooves 141a, 141b are provided on an outer circumferential wall of the inner portion 113a. The grooves 141a, 141b are separated by an apex 143. The grooves 141a, 141b are shaped so as to each be capable of receiving the head 131 of the cantilever 130. In this respect, FIG. 6A shows the head 131 residing in the first groove 141a, while FIG. 6B shows the head 131 residing in the second groove 141b. Upon rotation of the moveable member 111 within the limits defined by the switching tab 132 and slot 142, a portion of the cantilever 131 can flex outwards such that the head 131 may slide over the apex 143. For example, starting from the position shown in FIG. 6A, clockwise rotation of the moveable member 111 can move the head 131 from the first groove 141a, over the apex 143 and into the second groove 141b. As will be appreciated, an increased biasing force will be exerted by the cantilever 130 as the head 131 is moved over the apex 143. Therefore, once significant force is applied to the cantilever, the biasing force may cause the head 131 to snap into place in the adjacent groove. The same process can take place in reverse when starting from the position shown in FIG. 6B and rotating the moveable member 111 in the anticlockwise direction. Therefore, in addition to limiting the degree of movement of the moveable member 111, the connection hub 110 can be configured to provide two or more distinct switching positions. In this arrangement, when a user switches between the on and off positions, feedback is provided by virtue of the head 131 snapping into place within the first groove 141a or the second groove 141b.

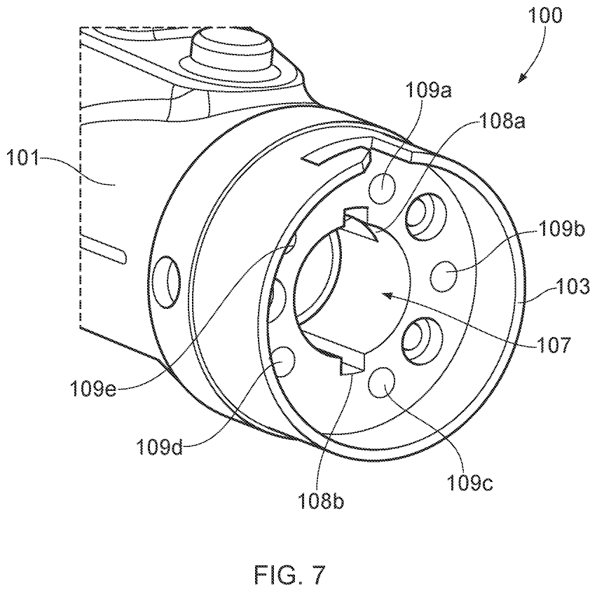

FIG. 7 shows further detail of the proximal end of the hand-piece 100, especially the guard member 103, without the collet 102. At an inner circumferential wall, the guard member 103 comprises at least one locking slot. In this arrangement, the guard member 103 comprises a first locking slot 108a and a second locking slot 108b at an opposite side of the chamber 107 to the first locking slot 108a. The locking slots 108a, 108b each provide a track extending axially through a portion of the hand-piece 100. The locking slots 108a, 108b are shaped so as to receive at least one corresponding locking tab. For example, the first locking slot 108a and a first locking tab 118a are shaped in a lock and key arrangement such that the locking tab 118 may slide along the locking slot 108a when these components are brought into alignment with one another. The first locking slot 108a may have different dimensions to that of the second locking slot 108b. For example, the first locking slot 108a may have a greater width than the second locking slot 108b. The hand-piece 100 further comprises at least one electrical contact. In this arrangement, five electrical contacts 109a, 109b, 109c, 109d, 109e are provided at an axial wall of the guard member 103. The electrical contacts 109a-109e can be arranged in a circular array about the chamber 107.

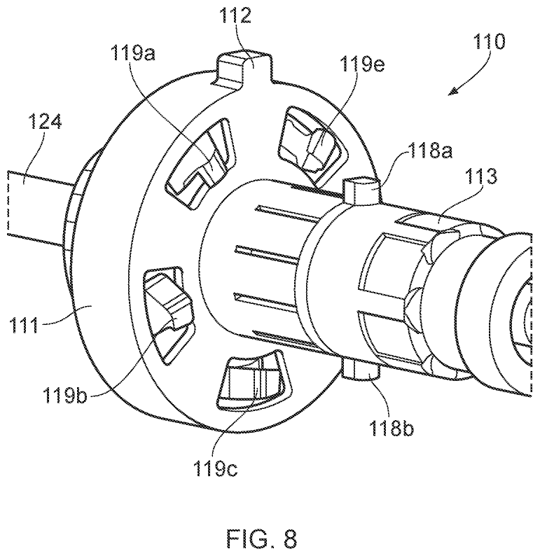

FIG. 8 shows further detail of the proximal end of an example of an electrosurgical instrument 120. FIG. 8 shows a view of the connection hub 110 from a direction opposite to that shown in FIG. 5. The static member 113 of the connection hub 110 comprises a first locking tab 118a and a second locking tab 118b. The locking tabs 118a, 118b may comprise radial projections of the static member 113 and may be disposed on opposite outer surfaces thereof. The first locking tab 118a may have different dimensions to that of the second locking tab 118b. For example, the first locking tab 118a may have a greater width than the second locking tab 118b. In this way, a ‘poka-yoke’ feature is provided wherein the connection hub 110 may only be inserted into the hand-piece when the first locking tab 118a is received by the first locking slot 108a and wherein the second locking tab 118b is received by the second locking tab 108b. In other words, the first locking tab 118a can be configured to be too big to fit into the second locking slot 108b. In this way, the device is configured to allow insertion of the electrosurgical instrument 120 into the hand-piece when the electrosurgical instrument 120 is in a single orientation. The required orientation may be that in which the first locking tab 118a is aligned with the first locking slot 108a and wherein the second locking tab 118b is aligned with the second locking slot 108b.

The moveable member 111 of the connection hub 110 further comprises at least one moveable electrical contact. In the example shown, the moveable member 111 comprises five moveable electrical contacts 119a-119e. The moveable electrical contacts 119a-119e may be configured in a circular array corresponding to that of the electrical contacts 109a-109e of the hand-piece 100. The moveable electrical contacts 1196a-119e are configured to contact the electrical contacts 109a-109e of the hand-piece 100. More specifically, the electrical contacts of the moveable member 111 are configured to contact the electrical contacts of the hand-piece 100 when the moveable member 111 is in its on position. The moveable electrical contacts 119a-119e are configured such that when the electrosurgical instrument 120 is inserted into the hand-piece 100 with the moveable member 111 in its off position, a first moveable electrical contact 119a is misaligned from a first electrical contact 109a on the hand-piece 100. Likewise, the second moveable electrical contact 119b is positioned so as to be misaligned from a second electrical contact 109b when the moveable member 111 is in its off position. The same is true for the remaining corresponding electrical contacts 109c-109e and moveable electrical contacts 119c-119e. A fourth moveable electrical contact is obscured from view in FIG. 8 but can be envisaged as being in a position corresponding to the electrical contact 109d of the hand-piece 100.

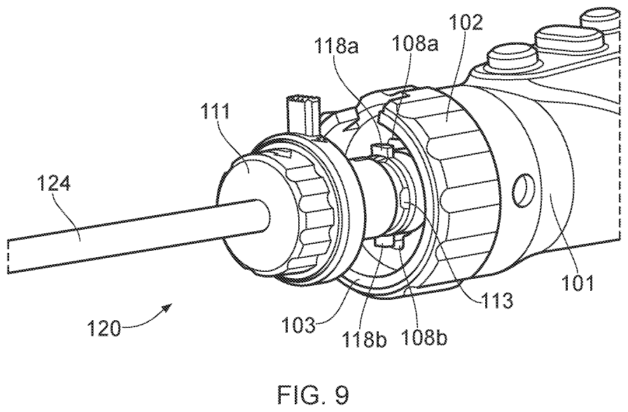

FIG. 9 shows the electrosurgical instrument 120 partially inserted into the hand-piece 100. In this illustration, the electrosurgical instrument 120 is positioned in a particular angular arrangement relative to the guard member 103 and the hand-piece 100. Specifically, the static member 113 of the connection hub 110 and the elongate shaft 124 are shown to be positioned relative to the hand-piece 100 such that the first locking tab 118a is rotationally aligned with the first locking slot 108a and such that the second locking tab 118b is rotationally aligned with the second locking slot 108b. Due to a lock-and-key arrangement between the locking slots and locking tabs, it will be understood that the connection hub 110 may only be inserted into the hand-piece 100 when each of the locking tabs is aligned with its corresponding locking slot. Should a user attempt to insert the electrosurgical instrument 120 at a different angle relative to the hand-piece 100 to that shown in FIG. 9, the guard member 103 can block the locking tabs from entering the chamber 107. In this way, the electrosurgical instrument 120 may only be inserted into the hand-piece 100 in a limited number of positions, the number being one in the illustrated arrangement.

Once the locking tabs 118a, 118b are partially inserted into their corresponding locking slots 108a, 108b, a locking member (not shown) may be provided to block the locking tabs 118a, 118b from sliding down a remainder of the locking slots 108a, 108b. The locking member can be fixed to the collet 102 such that it may rotate therewith relative to the guard member 103. In this arrangement, in order to temporarily unblock the locking slots 108a, 108b, the collet 102 may be rotated in a first direction relative to the hand-piece 100. The first direction may be clockwise in FIG. 9. Once the locking slots 108a, 108b are clear, the electrosurgical instrument 120 may be further inserted into the hand-piece 100. Once the locking tabs 118a, 118b are at an axial position further into the hand-piece 100 than the locking member, the collet 102 can be rotated in a second direction, opposite to the first direction, in order to re-introduce the locking member to block the locking slots 108a, 108b. In this way, the collet 102 can be used to open and close the locking slots 108a, 108b to lock the locking tabs 118a, 118b, and therefore lock the connection hub 110, into the hand-piece 100. In order to remove the locking tabs 118a, 118b from the locking slots 108a, 108b, the collet 102 must again be turned in the first direction in order to unblock the locking slots 108a, 108b to permit the connection hub 110 to be disconnected from the hand-piece 100.

The collet 102 may comprise a biasing member (not shown) to bias the locking member towards an equilibrium position such as that shown in FIG. 9. In this way, when the collet 102 is rotated in the first direction to allow for entry of the locking tabs 118a, 118b into the locking slots 108a, 108b, releasing the collet 102 can allow the biasing member to bias the collet 102 and the locking member towards the second direction. This provides a default position of the collet 102 and locking member in which the locking slots 108a, 108b are blocked in order to secure the electrical instrument 120 to the main body 101 of the hand-piece 100. It will be understood that once the locking tabs 118a, 118b are received by the corresponding locking slots 108a, 108b, the electrosurgical instrument 120 is restricted from rotation about the longitudinal axis thereof. In particular, the elongate shaft 124 and the connection hub 110 are restricted from rotation relative to the hand-piece 100 when the locking tabs 118a, 118b are located in the locking slots 108a, 108b.

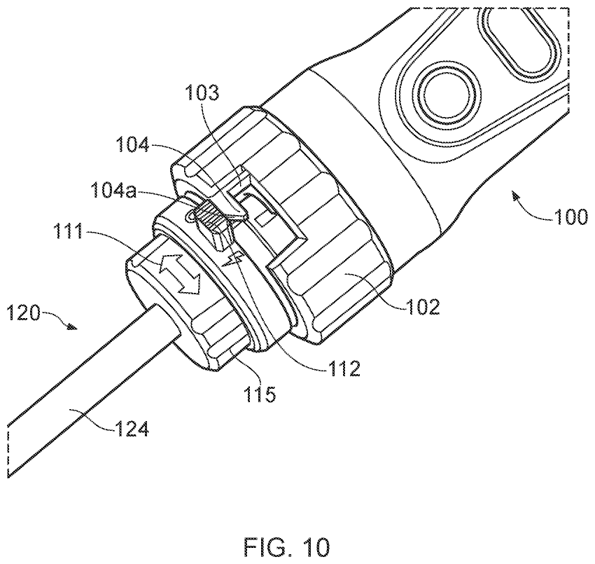

FIG. 10 shows the electrosurgical device wherein the electrosurgical instrument 120 is shown to be partially inserted into the hand-piece 100. The electrosurgical instrument 120 is shown to be located at an axial position relative to the hand-piece that is representative of the locking tabs 118a, 118b being positioned within the locking slots 108a, 108b. Therefore, the elongate shaft 124 is restricted from rotating relative to the hand-piece 100. In FIG. 10, the moveable member 111 is shown to be in its on position. The result of the electrosurgical instrument 120 being introduced into the hand-piece 100 while the moveable member 111 is in its on position is that the guard tab 104 blocks the arm 112, and likewise blocks the electrosurgical instrument 120 as a whole, from advancing axially further into the hand-piece 100. As disclosed above, when the moveable member 111 is in the on position at the time of partial insertion of the electrosurgical instrument 120 into the hand-piece 100, the moveable electrical contacts 119a-119e are aligned, but not in contact with, the electrical contacts 109a-109e of the hand-piece 100. Therefore, without the presence of the guard member 104, sliding the electrosurgical instrument 120 further into the hand-piece 100 while the moveable member 111 is in the on position would result in the moveable electrical contacts 119a-119e coming into contact with the electrical contacts 109a-109e of the hand-piece 100. In this situation, were any of the inputs of the electrosurgical device to be activated, for example by operation of a button, inserting the electrosurgical instrument in the on position could lead to the device being in a live state in which any of the surgical features, such as shaving, are activated. In other words, with the moveable member in the on position, complete insertion into the hand-piece could result in accidental activation of the surgical operations. Even were such surgical features deactivated, inserting the electrosurgical instrument in the on position could result in damage to the electrical components. Instead, to prevent these disadvantageous outcomes, the arrangement of the guard tab 104 and the arm 112 can prevent the electrosurgical instrument 120 being fully inserted into the hand-piece 100 when the moveable member 111 is in its on position. The arrangement ensures that the electrosurgical instrument 120 can only be fully inserted into the hand-piece 100 when in its off position. Therefore, even were the inputs of the hand-piece to be activated, for example by accidentally pushing one of the buttons during insertion of the electrosurgical instrument, the moveable member being in its off position would prevent the electrosurgical device being put into a live state.

From the position shown in FIG. 10, in order to connect the electrosurgical instrument 120 to the hand-piece 100, the moveable member 111 must be switched to its off position. This may be done by rotating the moveable member, for example by applying a force to the arm 112 or to a circumferential knurled surface 115 of the moveable member 111. The guard tab 104 may comprise a sloped edge 104a. In the example shown, the sloped edge 104a slopes in a direction that lies in the plane of the annular portion of the guard member 103. The sloped edge 104a can be configured as a cam such that when the electrosurgical instrument 120 is axially forced towards the hand-piece 100, the arm 112, acting as a cam follower, can slide along the sloped edge 104a to thereby switch the moveable member 111 from its on position to its off position. Whichever method is used to switch the moveable member 111 to its off position, once the moveable member 111 is in its off position, the arm 112 will no longer be obstructed from axial movement by the guard tab 104 and so the electrosurgical instrument 120 can be full inserted into the hand-piece 100, to the position shown in FIG. 2. The moveable member 111 can then be switched to its on position, as shown in FIG. 3, in order to align and connect the moveable electrical contacts 119a-119e with the electrical contacts 109a-109e to thereby provide an electrical connection between the electrosurgical instrument 120 and the hand-piece 100. Then, by operation of the user inputs or otherwise, the surgical features can be activated to safely put the device into a live state.

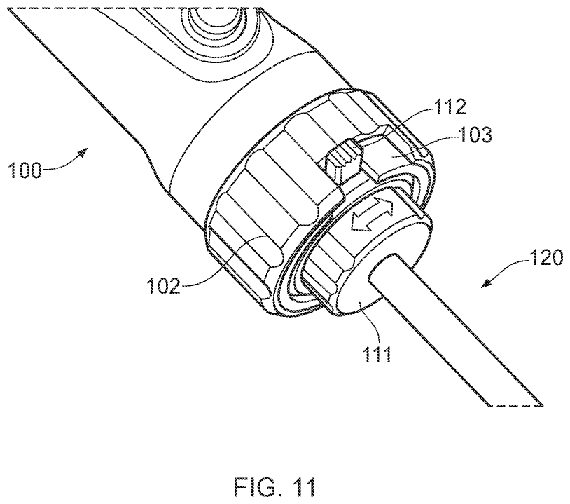

FIG. 11 shows the electrosurgical device wherein the electrosurgical instrument 120 is in the process of being removed from the hand-piece 100. From the fully inserted and on position shown in FIG. 3, the electrosurgical instrument 120 may be removed by: firstly, switching the moveable member 111 to the off position, for example by actuating the arm 112; secondly, rotating the collet 102 in the first direction to remove the locking member from blocking the locking slots 108a, 108b; and thirdly, moving the electrosurgical instrument 120 away from the hand-piece 100 while holding the collet 102 in the unlocked position. Alternatively, the first two of these steps can be performed in one movement by: rotating the collet 102 in the first direction (clockwise in FIG. 11) such that a window edge of the collet 102 actuates the arm 112 to switch the moveable member 111 to its off position and then continuing to rotate the collet 102 in the first direction in order to unblock the locking slots 108a, 108b. The relative positions of the collet 102, arm 112 and guard member 103 after this step are illustrated in FIG. 11. After this step, the electrosurgical instrument 120 can be removed from the hand-piece 100 as before.

Overall, the electrosurgical system disclosed herein provides an electrosurgical instrument 120 that can be connected, in two stages, to a hand-piece 100 for provision of electrosurgical signals, mechanical power, suction and/or irrigation, among other surgical operations. The first stage of connection dictates a necessary condition that the electrosurgical instrument 120 be in a particular orientation relative to the hand-piece before insertion can take place. As described above, the first stage of connection can be provided by an arrangement of locking tabs 118a, 118b and locking slots 108a, 108b on the electrosurgical instrument 120 and the hand-piece 100 respectively, or vice versa. The first stage of connection ensures that the electrosurgical instrument is inserted into the hand-piece the correct way round, i.e. in the correct orientation. The second stage of connection dictates a further necessary condition that a switch of the electrosurgical instrument be in a particular position before insertion can take place. As described above, the second stage of connection can be provided by an arrangement of a moveable member 111 and a guard tab 104 on the electrosurgical instrument 120 and the hand-piece respectively, or vice versa. The second stage prohibits insertion of the electrosurgical instrument 120 into the hand-piece 100 when the switch is in the on position. In this way, the second stage of connection reduces the likelihood of damage to the electrical circuits and prevents accidental activation in situations in which the electrosurgical instrument 120 is inserted into the hand-piece 100 while one or more of the mechanical, fluidic or electrical inputs are in an activated position.

While the present disclosure refers to electrosurgical instruments, devices and systems, it will be appreciated that the disclosure is also applicable to any surgical instrument, device or system having an electrically powered end effector connectable to a hand-piece. In other embodiments, the arrangement can also be applicable to end effectors which are powered by other modes, such as pneumatically or hydraulically.

Although the arrangement has been described with reference to a substantially circular arrangement, a similar arrangement for a different shape could be envisaged. For example, the connection hub may have a substantially rectangular cross section, receivable by a correspondingly rectangular channel in a hand-piece. Instead of the moveable member being rotatable with respect to the elongate shaft, it may be laterally translatable. In line with this arrangement, the collet and guard member may comprise substantially rectangular portions in order to control the access of the moveable member into the hand-piece based on the lateral position of the switch. Such an arrangement, or similar arrangements, can provide the same benefits of a two stage connection as described above.

While the embodiments described above and illustrated in the figures have included a specific number of components, such as locking slots, locking tabs and electrical contacts, it will be understood that the disclosure is not limited to these specific examples. In particular, an electrosurgical device of an alternative embodiment may have a single locking slot and corresponding locking tab, or it may have more than two sets of corresponding locking slots and locking tabs. Similarly, the hand-piece may have fewer or greater than five electrical contacts configured to contact a corresponding number of moveable electrical contacts on the connection hub. Further, while a single arm and guard tab has been described, alternative embodiments may adopt more than one set of these components to provide the second stage of connection.

Various modifications, whether by way of addition, deletion and/or substitution, may be made to all of the above described embodiments to provide further embodiments, any and/or all of which are intended to be encompassed by the appended claims.

1. A surgical instrument, configured to be releasably connectable to a hand-piece, the surgical instrument comprising:
an elongate shaft,
an end effector at a distal end of the elongate shaft, and
a connection hub at a proximal end of the elongate shaft, the connection hub comprising a moveable member, moveable relative to the elongate shaft, and configured to be switchable between an on position and an off position,
configured such that during insertion of the surgical instrument into the hand-piece, an electrical connection therebetween is prevented by the connection hub when the moveable member is in the on position.

2. The surgical instrument according to claim 1, wherein the moveable member comprises an arm which, in combination with a guard tab of the hand-piece, is configured to prevent insertion of the surgical instrument into the hand-piece when the moveable member is in the on position to thereby prevent an electrical connection between the surgical instrument and the hand-piece.

3. The surgical instrument according to claim 1, wherein the connection hub comprises at least one electrical contact configured to be aligned with at least one corresponding electrical contact on the hand-piece when the moveable member is in the on position.

4. The surgical instrument according to claim 3, configured such that after insertion of the surgical instrument into the hand-piece, switching the moveable member from the off position to the on position brings at least one electrical contact of the connection hub into alignment with at least one corresponding electrical contact of the hand-piece, to thereby permit electrical connection therebetween.

5. The surgical instrument according to claim 1, wherein the connection hub further comprises a static member in fixed relation to the elongate shaft, the static member comprising at least one locking tab configured to be received by at least one locking slot of the hand-piece.

6. The surgical instrument according to claim 1, the moveable member comprising a cantilever, the cantilever comprising a cantilever head, and the static member comprising a first groove and a second groove, each one of the first and second grooves being configured to receive the cantilever head, configured such that switching the moveable member between the on position and the off position moves the cantilever head from the first groove to the second groove in order to provide two distinct switching positions of the moveable member.

7. A surgical instrument according to claim 1, wherein the surgical instrument is an electro-surgical instrument.

8. A hand-piece, configured to power a surgical instrument electrically, the hand-piece comprising:
a main body, and
a guard member in fixed relation to the main body, the guard member comprising a guard tab configured to prevent insertion of a moveable member of the surgical instrument into the hand-piece when the moveable member is in an electrically on position,
wherein the moveable member is switchable between the on position and an off position.

9. The hand-piece according to claim 8, wherein the guard tab comprises a cam surface configured to direct the moveable member of the surgical instrument towards the off position.

10. The hand-piece according to claim 8, further comprising a collet rotatable with respect to the guard member and the main body between a locked and an unlocked position, configured to prevent insertion of the surgical instrument into the hand-piece when in the locked position.

11. The hand-piece according to claim 8, wherein the collet is configured to switch the moveable member to its off position when the collet is rotated in a first direction.

12. The hand-piece according to claim 11, wherein the collet is configured to switch the moveable member to its on position when the collet is rotated in a second direction, opposite the first direction.

13. A surgical system comprising a surgical instrument and a hand-piece, the surgical instrument being configured to be releasably connectable to a hand-piece, the surgical instrument comprising:
an elongate shaft, an end effector at a distal end of the elongate shaft, and a connection hub at a proximal end of the elongate shaft, the connection hub comprising a moveable member, moveable relative to the elongate shaft, and configured to be switchable between an on position and an off position, configured such that during insertion of the surgical instrument into the hand-piece, an electrical connection therebetween is prevented by the connection hub when the moveable member is in the on position;
the handpiece being configured to power the surgical instrument electrically, the hand-piece comprising:
a main body, and a guard member in fixed relation to the main body, the guard member comprising a guard tab configured to prevent insertion of a moveable member of the surgical instrument into the hand-piece when the moveable member is in an electrically on position, wherein the moveable member is switchable between the on position and an off position.

14. The surgical system according to claim 13, and further comprising a radiofrequency electrosurgical generator, the surgical system being an electrosurgical system.

15. The surgical system according to claim 14, wherein the radiofrequency electrosurgical generator is comprised within the hand-piece as an integral part thereof.

16. A surgical device comprising:
a hand-piece;
a surgical instrument comprising an elongate shaft and an end effector, the surgical instrument configured to be releasably connectable to the hand-piece; and
a mutually cooperating interface between the hand-piece and the surgical instrument configured to prevent insertion of the surgical instrument into the hand-piece in an electrically live state.

17. A surgical device according to claim 16, wherein the surgical device is an electro-surgical device.
